(19) United States
(12) Patent Application Publication
(10) Pub. No.: US 2005/0178392 A1
(43) Pub. Date: Aug. 18, 2005

(54) Device to reduce snoring, drooling, and talking while sleeping

(76) Inventor: Thomas Austin Tinsley, New Port Richey, FL (US)

Correspondence Address: THOMAS A. TINSLEY, 7337 OTTER CREEK DRIVE, NEW PORT RICHEY, FL, 34655, US

(21) Appl. No.: 10/778,510

(22) Filed: Feb. 16, 2004

(51) Int. Cl. A61F 5/56

(52) U.S. Cl. 128/848

(57) ABSTRACT

This device reduces snoring, drooling, or talking during sleep. A small piece of cloth tape or other porous hypoallergenic material with a hypoallergenic adhesive on the back is affixed to the lips before sleeping. This small piece of tape operates like a latch to keep the mouth shut and prevents the wearer from breathing through their mouth. The wearer of the device may easily remove the device to talk, sneeze, cough, yawn, etc. when needed. The device is small, can be worn with a mustache or beard, may have a message printed on the front, may be made in various sizes and shapes, and may be a color besides white. It is packaged with a protective backing that is peeled off before using the device.

### CROSS REFERENCE TO RELATED APPLICATIONS

### U.S. Patent Documents

| | | | |
|---|---|---|---|
| 6089232 | July, 2000 | Portnoy | 128/848 |
| 5810013 | September, 1998 | Belfer | 128/848 |
| 5642737 | July, 1997 | Parks | 128/848 |
| 4817636 | April, 1989 | Woods | 128/848 |
| 6675804 | January, 2004 | Pivovarov | 128/848 |
| 6619290 | September, 2003 | Zacco | 128/848 |
| 6601585 | August, 2003 | Conrad, et al. | 128/898 |
| 6526982 | March, 2003 | Strong | 128/848 |
| 6467485 | October, 2002 | Schmidt | 128/848 |
| 6427696 | August, 2002 | Stockhausen | 128/848 |
| 6412489 | July, 2002 | Sue | 128/848 |
| 6263877 | July, 2001 | Gall | 128/848 |
| 5988170 | November, 1999 | Thomas | 128/848 |
| 5893365 | April, 1999 | Anderson | 128/848 |
| 5884628 | March, 1999 | Hilsen | 128/848 |
| 5823193 | October, 1998 | Singer | 128/848 |
| 5715840 | February, 1998 | Hall | 128/848 |
| 5687743 | November, 1997 | Goodwin | 128/848 |
| 5642738 | July, 1997 | Lilly, Fr. | 128/848 |
| 5566683 | October, 1996 | Thornton | 128/848 |
| 5499633 | March, 1996 | Fenton | 128/848 |
| 5316020 | May, 1994 | Truffer | 128/848 |
| 5267862 | December, 1993 | Parker | 443/215 |
| 5117816 | June, 1992 | Shapiro, et al. | 128/200.24 |
| 5092346 | March, 1992 | Hays, et al. | 128/848 |
| 5046512 | September, 1991 | Murchie | 128/848 |
| 4669459 | June, 1987 | Spiewak, et al. | 128/848 |

### BACKGROUND OF THE INVENTION

Individuals that snore, drool, or talk in their sleep have a social problem as well as a potential medical problem.

The following extract from an article, “Snoring: Not Funny; Not Hopeless” from Medline Plus Health Information, a service of the U.S. National Library of Medicine and the National Institute of Health, sums up the problem as:
Forty-five percent of normal adults snore at least occasionally, and 25 percent are habitual snorers. Problem snoring is more frequent in males and overweight persons, and it usually grows worse with age.
More than 300 devices are registered in the U.S. Patent and Trademark Office as cures for snoring. Some are variations on the old idea of sewing a sock that holds a tennis ball on the pajama back to force the snorer to sleep on his side. (Snoring is often worse when a person sleeps on his back). Some devices reposition the lower jaw forward; some open nasal air passages; a few others have been designed to condition a person not to snore by producing unpleasant stimuli when snoring occurs. But, if you snore, the truth is that it is not under your control whatsoever. If anti-snoring devices work it is probably because they keep you awake.

In this same article the following answers show that snoring is a problem:
Socially, yes! It can be, when it makes the snorer an object of ridicule and causes others sleepless nights and resentfulness.
Medically, yes! It disturbs sleeping patterns and deprives the snorer of appropriate rest. When snoring is severe, it can cause serious, long-term health problems, including obstructive sleep apnea.

Today there are many products available to address this serious problem. There are
Sprays to help numb the soft tissue in the throat,
Special pillows to prop up the head or prevent someone from sleeping on their back,
Electrical shocks when a device recognizes someone snoring,
Devices to help open the nasal passages,
Devices to insert in the mouth, and
Large devices to strap on the face.

From the medical perspective, there are lifestyle changes that can be made such as losing weight and medical procedures to modify an individual's air flow passage to curb snoring.

In summary, getting a good night's sleep for the individual sleeping, and for their sleeping partners, is a serious issue that demands that there be many options available to satisfy specific individual needs. In addition, there must be socially acceptable devices that could be used while sleeping on public transportation or other group settings.

### BRIEF SUMMARY OF THE INVENTION

The purpose of this invention is to reduce snoring, drooling, and talking. During sleep, air flows in and out through the nasal passage and through the oral cavity. Although an increase in the airflow through the nasal passage may significantly reduce snoring, stopping the airflow through the oral cavity should eliminate most snoring. As an added benefit, this will also prevent drooling and talking during sleep.

Snoring is palatal snoring or base of tongue snoring. The vibration of the uvula in the back of the throat causes palatal snoring. Base of tongue snoring is caused by restricted space behind the tongue, which causes turbulence as the air flows through. In both of these cases, a single flow of air through the nasal passage should not cause the same noise as that caused by airflow through the oral cavity.

This invention is designed to utilize a small piece of tape to pull the lips together. The tape would be hygienically sealed and the seal would be removed before use. Application of the tape should be easy for any adult. The tape would only cover the center part of the lips and operate as a latch to keep the mouth shut rather than a piece of tape across the entire mouth. Approximately 1/3 of the lips would be taped.

The tape would be applied when settling down for sleep and removed when arising from sleep. Applying the device would be done in the following order:
1. Remove the non-stick material on the back of the tape
2. Press the top part of the device to the upper lip
3. Gently pull the tape from the bottom using the tab to pull the lips together
4. Press the tape to the bottom lip.

The advantages of this device include:
A socially acceptable appearance,
It is non-invasive,
It is non-chemical, and
A person can sleep in any position while wearing the device.

### BRIEF DESCRIPTION OF THE SEVERAL VIEWS OF THE DRAWINGS

FIG. 1 This side view shows how the tape is placed on the lips to prevent airflow through the oral cavity. This allows for airflow to occur primarily through the nasal cavity.
10 Nasal cavity.
20 Oral cavity.
30 Device affixed to the lips.

FIG. 2 This front view shows the approximate shape of the device.

FIG. 3A This back view shows the device and how a layer is removed to expose the adhesive-covered surface shown in FIG. 3B.
10 The first layer on the back of the device peels off and is removed.
20 The second layer with adhesive on the surface.

FIG. 3B This back view shows how the adhesive on the back of the device follows the shape of a pair of lips but provides a tab for applying and removing the tape.
10 Adhesive on the device in the general shape of a pair of lips.
20 An adhesive-free tab for affixing and removing the device.

### DETAILED DESCRIPTION OF THE INVENTION

The device is produced from hypoallergenic tape. This type of tape is used for attaching bandages or wrapping sprained ankles and wrists. This type of tape allows for the skin to breath. As shown in FIG. 1, the tape would only touch the lips.

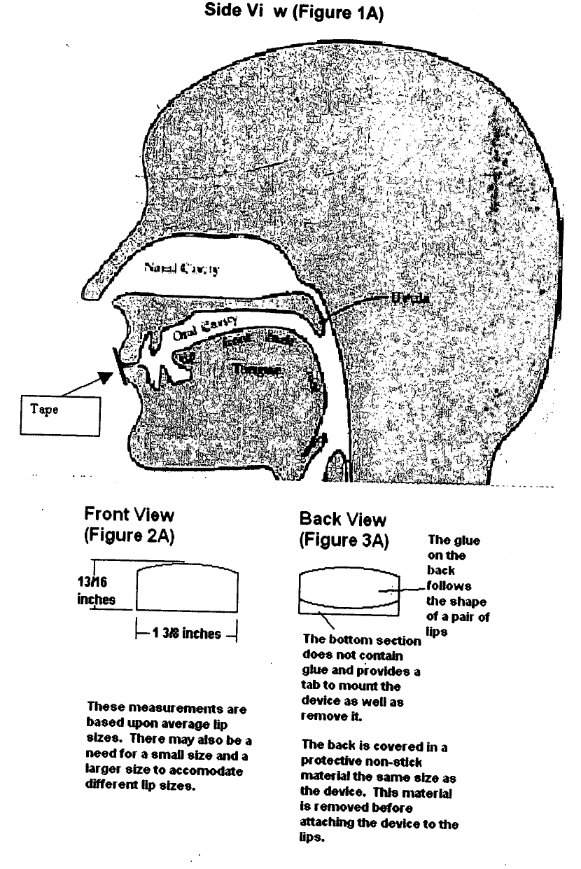

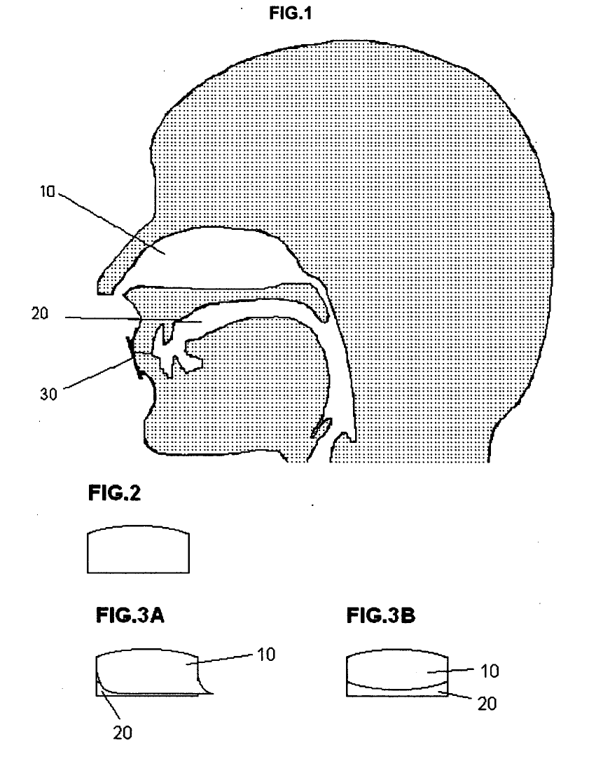

As shown in FIG. 2, the tape is cut to the general shape of an upper lip at the top and cut straight at the bottom. On the back of the tape, as shown in FIG. 3B, a tab is created by covering the bottom of the tape to cover the adhesive on the tape so the area with adhesive generally represents the shape of a pair of lips. Then, a non-stick material is placed on the back of the device to cover both the adhesive area and the tab. Where the non-stick material covers the tab, the non-stick material can be easily pulled up for removal as shown in FIG. 3A.

1. This device reduces a person's potential of snoring while sleeping by preventing the flow of air between the lips by affixing an adhesive-backed tape to the upper and lower lips.

2. The adhesive on the back of the device by claim 1 is covered with a protective backing that is removed before placing the device on the lips.

3. This device reduces sleep apnea by claim 1.

4. This device prevents a person from drooling while sleeping by holding the mouth closed.

5. This device prevents a person from clarity in talking by restraining the movement of their lips while sleeping.

6. The device is in the basic shape of a pair of lips so that it is only affixed to the lips and not the skin around the lips.

7. The size of the device is based upon average lip sizes and may come in different sizes such as small, medium, large, and extra-large.

8. This device is hypoallergenic.

9. This device has a non-adhesive tab on one of the edges that allows for quick and easy removal.

10. The tab in claim 9 may be used to pull this device off one lip to allow for normal use of the lips for activities such as sneezing, talking, yawning, coughing, etc. At the completion of the activity, this device may be affixed to the lip again.

11. This device may have printed messages on the non-adhesive front side such as “Do not disturb” to provide social acceptability while wearing this device.

12. This device may be constructed with different
Colors and
Shapes.
